(19) United States
(12) Patent Application Publication
(10) Pub. No.: US 2006/0172425 A1
(43) Pub. Date: Aug. 3, 2006

(54) Colored buffer solution for automated clinical analyzer

(75) Inventors: Ralf Neigl, Yorktown Heights, NY (US); Michael J. Sommer, Monroe, NY (US); Bronislaw P. Czech, Cortlandt Manor, NY (US); Horst Berneth, Leverkusen (DE); Alan R. Toth, Newburgh, NY (US); Josef-Walter Stawitz, Odenthal (DE)

Correspondence Address: RODMAN RODMAN, 7 SOUTH BROADWAY, WHITE PLAINS, NY, 10601, US

(73) Assignee: BAYER HEALTHCARE, LLC, Tarrytown, NY (US)

(21) Appl. No.: 11/048,601

(22) Filed: Feb. 1, 2005

(51) Int. Cl. G01N 33/00

(52) U.S. Cl. 436/18

(57) ABSTRACT

A colored intercapsular buffer solution is used to reduce erroneous results from the absorption or carryover of small amounts of reagent R1 and/or R2 into the intercapsular buffer solution segment in the analytical line of a capsule chemistry liquid analysis system in an automated clinical analyzer. The absorption of small amounts of the regent R1 and/or R2 in the intercapsular buffer solution leads to erroneous results in the analysis of the test sample, S, because the R1 and/or R2 that is carried over and absorbed into the buffer does not react with test sample S. The R1/R2 carryover can be measured and determined by monitoring the absorbance of the colored buffer solution, wherein the change in absorbance can be measured and compared to a reference value. The sample is automatically retested if an unacceptable change in absorbance occurs. The colored buffer solution comprises a phthalocyanine dye, an aqueous solution of a base at a pH of about 9-12, a surfactant to reduce surface tension and improve flow characteristics of the intercapsular colored buffer reagent through the analytical line, and a chelating agent to reduce the adverse effects of metal contaminants.

### BACKGROUND OF THE INVENTION

This invention relates to capsule chemistry sample liquid analysis systems for the automated clinical analysis of biological fluid samples, such as blood.

The entire analytical process, from sampling to readout, occurs in a single capillary tube, also referred to as the analytical line or conduit. A typical automated capsule chemistry liquid analysis system is described in U.S. Pat. No. 5,268,147 to Zebetakis et al, the disclosure of which is incorporated by reference herein.

The analytical system is hydraulic in nature and utilizes oil isolation liquid IL, a colorless intercapsular buffer B, sample S, reagent R1, and reagent R2, wherein the objective is to form a test composition R1SR2 for automated clinical analysis.

Hydraulic abnormalities sometimes occur in the analytical line due to the absorption or carryover of a small amount of R1 or R2 in the intercapsular buffer B, which can lead to an incomplete R1SR2 test composition and erroneous test results.

### BRIEF DESCRIPTION OF THE DRAWINGS

FIG. 1 is a simplified schematic representation of the principal features of an automated capsule chemistry sample liquid analysis system;

FIG. 2 is an enlarged schematic representation of a portion of the analytical line showing a test package before and after passing through the vanish zone of the sample liquid analysis system.

### DESCRIPTION OF THE PREFERRED EMBODIMENTS

The present invention improves the performance of automated clinical analyzers and reduces hydraulic abnormalities and erroneous results that occur when a small fraction of the reagent R1 or reagent R2 is absorbed by the intercapsular buffer segment. When this occurs, the total amount of R1 and/or R2 is not used to form the R1SR2 test composition.

This invention is particularly adaptable to the ADVIA IMS™ chemistry module clinical analyzer (Bayer HealthCare LLC) and can also be used with other clinical analyzers.

The inventive method reduces erroneous results in the capsule chemistry liquid analysis system by using an intercapsular colored buffer solution in place of the colorless intercapsular buffer solution. Erroneous results occur from the absorption or carryover of small amounts of reagent R1 and/or R2 into the intercapsular buffer solution segment in the analytical line of an automated clinical analyzer. This is because the R1 and/or R2 that is carried over and absorbed into the buffer does not react with the test sample S.

The amount of R1 or R2 that becomes absorbed in the intercapsular colored buffer solution can be monitored by measuring the change of absorbance in the colored buffer solution segment of each test package and comparing it to a reference value. The sample is reanalyzed if an unacceptable change in absorbance has occurred.

In operation, the clinical analyzer registers the change in absorbance of the dye in the colored buffer solution. The absorbance of the buffer segment is measured to establish a baseline or threshold measurement, before the reagent segments merge. After the regent segments have merged, the absorbance of the buffer segment is again measured.

An unacceptable change in absorbance will occur when the threshold absorbance of the buffer segment has been exceeded. An error flag will then be produced on the ADVIA IMS clinical analyzer. The specific threshold values are determined empirically for each individual assay.

The threshold absorbance is a determination that is based on the sensitivity of the specific assay to the loss of reagent. When the threshold is exceeded, the results are flagged and are not reported to the end user. The sample is also flagged to be automatically retested so that accurate results can be generated.

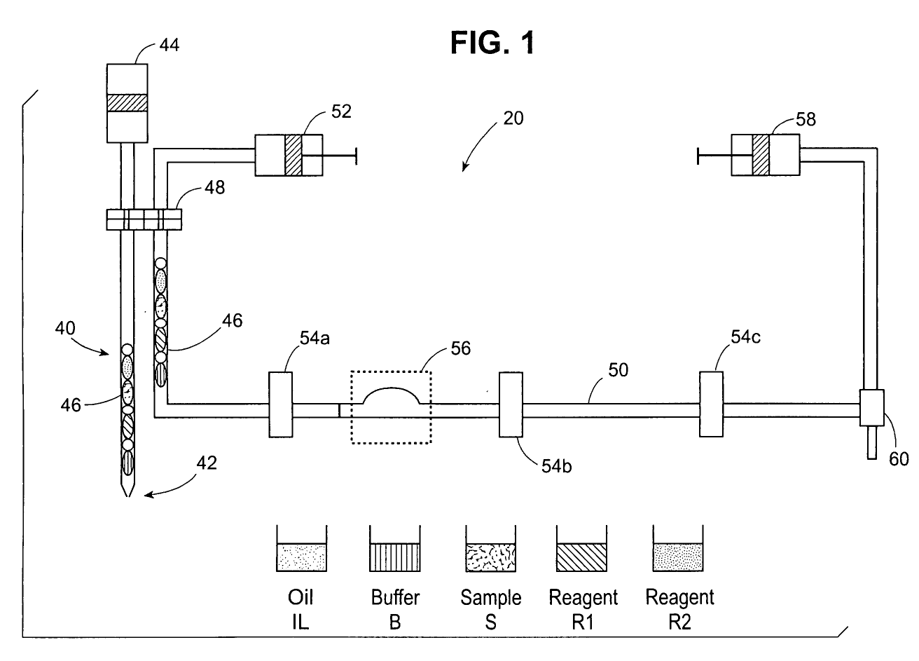

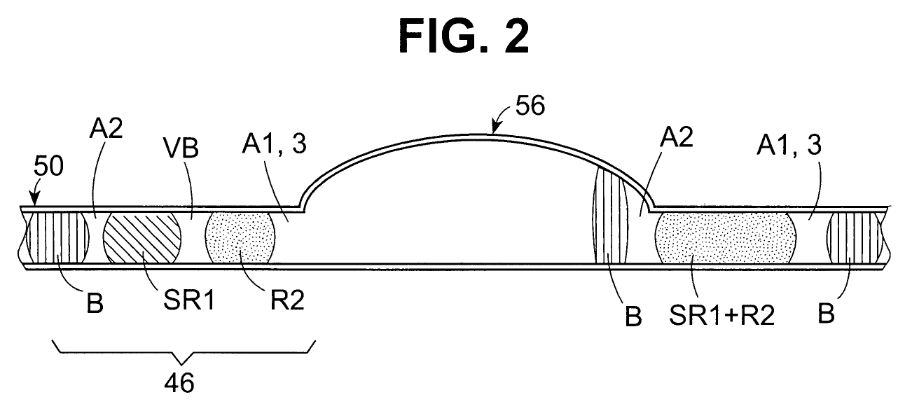

The automated clinical analysis system contains a long, narrow, optically clear capillary tube preferably made of Teflon® (DuPont Co.) or like material with pumps at the near end and far end. Referring to FIGS. 1 and 2, the automated clinical analysis system 20 comprises a sample liquid test package aspirating assembly 40 with a probe 42 and a pump 44 that is used to aspirate the liquid portion or aliquots of the test segments comprising the test package 46. These liquid portions include the sample S, the aqueous reagent aliquots R1 and R2, the intercapsular buffer solution B, and the oil isolation liquid, IL, which are shown more clearly in FIG. 2. The intercapsular buffer solution B is used to separate test packages in the analysis system.

A shear valve 48 serves to transfer the test package 46 to the analytical line 50 where pump 52 transfers the test package 46 through flow cell 54a. Flow cell 54a is used to read the optical absorbance of liquid reagents R1 and R2 before reagents R1 and R2 merge in vanish zone 56.

FIG. 2 shows an enlarged portion of the analytical line 50 with the vanish zone 56 of FIG. 1. Sample S and reagent R1 merge immediately inside the probe 42 upon aspiration to produce the reagent/sample capsule SRI. The aliquots SR1 and R2 are separated by an air segment VB, referred to as the vanish bubble. An aqueous intercapsular buffer segment B, interposed between the two air segments, A2 and A1, 3 respectively, is used to separate different test packages inside the analytical line 50.

After each aspiration of one test package 46, one previously aspirated test package 46 is transferred past the shear valve 48 and introduced into the analytical line 50. A “push-pull” pumping mechanism is initiated which transports the test package 46 in the analytical line 50 in a back and forth motion. This back and forth motion allows each test segment in the test package 46 several opportunities for its optical properties to be read by the flow cells 54b and 54c. Flow cells 54b and 54c read the optical absorbance of test sample/first reagent SR1, and second reagent R2 at different times after merging and passing through vanish zone 56. After each cycle, which consists of one aspiration of a test package and one back and forth motion, the next test package will be introduced into the analytical line 50. After the analysis has been completed, pump 58 disposes unwanted test package materials to waste collection 60.

The configuration and structure of the reaction capsules SR1 and R2 is influenced by the isolation liquid, IL, which wets and coats the hydrophobic inner surface of the analytical line 50 with a thin, flowing film of the isolation liquid IL. The isolation liquid is replenished continuously as new samples are aspirated into the analytical line 50.

The isolation liquid IL that coats the inner walls of the analytical line 50 is typically a fluorocarbon or silicon liquid, such as FC43® (3M Co.), FC70® (3M Co.), and DC 200® (Sigma-Aldrich Co.). The isolation liquid IL prevents contact of the liquid test packages that flow through the analytical line 50 with the inner surface of the analytical line and is immiscible with the sample, reagent and intercapsular buffer liquids that comprise each test package. Thus, the isolation liquid IL substantially and completely excludes any residual presence or carryover of the sample, reagent, and buffer liquids on the inner surface of the analytical line 50.

As noted, the role of the intercapsular buffer solution whether colored or not is to separate test packages in the analytical line. Typical intercapsular buffer solutions contain suitable bases such as alkali hydroxide, preferably sodium hydroxide, potassium hydroxide and other equivalents. The function of the hydroxide is to increase the pH of the buffer to about 8 to 13, preferably to a pH of about 9 to 12, and to help stabilize the dye.

It has been found that the incorporation of a dye into the intercapsular buffer solution facilitates the monitoring and measuring of the amount of reagent R1 and/or R2 that can carryover and become absorbed in the intercapsular buffer solution segment. The monitoring and measurement is accomplished by use of the flows cells 54a, 54b, and 54c, as noted in FIG. 1, to measure the change in the absorbance of the buffer solution segment. Thus, the amounts of R1 or R2 that become carried over or absorbed into the colored buffer solution will dilute the dye, resulting in a decrease in absorbance. Reagents R1 and R2 by themselves have no significant absorbance at 600 nm.

The suitable dyes that can be used to form the intercapsular colored buffer solution must be stable at alkaline pH and demonstrate maximum absorbance near 600 nanometers (“nm”). Thus, for example, if the dye is scanned in a spectrophotometer, the highest optical density will occur at 600 nm. A suitable dye should also be stable to changes in pH. pH stability usually occurs with aggregated dye species, that is, when the identical molecules attach to themselves to form dimers and trimers. Suitable dyes include the phthalocyanine dye group, preferably sulfonic acid phthalocyanine dyes and more preferably copper phthalocyanine sulfonic acid dyes, such as Pontamine Brilliant Blue® (Bayer Corporation), Bayscript Cyan Blue (Bayer Corporation), Pontamine Substantive Turquoise (Bayer Corporation), and copper (II) phthalocyanine-3,4′,4″,4′″-tetrasulfonic acid.

The effective amounts of dye in the intercapsular colored buffer solution can vary from about 1 mg/liter to about 10 mg/liter, preferably from about 4.5 mg/liter to about 7.5 mg/liter, and most preferably from about 5 mg/liter to about 7 mg/liter.

Again, referring to FIGS. 1 and 2, by way of illustration, about 7 μl of the intercapsular colored buffer solution is aspirated into the analytical line 50. R1 and R2 are also aspirated as separate segments into the stream. An air bubble separates each segment. As R1 and R2 flow through the analytical line 50, a small volume of either R1, R2 or both R1 and R2 on the order of about 0.5 μl is carried over into the intercapsular colored buffer solution, thereby diluting the dye. The change in color due to the presence of R1 and/or R2 is registered as a decrease in absorbance by flow cells 54a, 54b and 54c on the ADVIA IMS clinical analyzer (Bayer Corporation).

The intercapsular colored buffer solution also includes a suitable surfactant to reduce surface tension of the intercapsular buffer solution and to improve the flow of the test composition in the analytical line. Suitable surfactants include, for example, linear alcohol alkoxylates such as Plurafac RA-20®, RA-30®, RA-40®, RA-43® (BASF Corporation), Brij 35 (Atlas Chemical Co.) and the like, or a non-ionic alkylaryl polyether alcohol, such as Triton X-100® (Rohm & Haas Co.), and the like.

The surfactant concentration can vary in amounts from about 0.3% (w/v) to about 3.0% (w/v), preferably about 0.5% (w/v) to about 2% (w/v) and most preferably about 1% (w/v). Most preferably, the intercapsular colored buffer solution contains about 50 mM sodium hydroxide and about 1% (w/v) of suitable surfactant.

Although phthalocyanine dyes are well known for their stability in both alkaline and acidic media, stability studies of the intercapsular colored buffer solution showed significant color degradation after about 1-2 months, especially at elevated temperatures of about 30° C. to about 50° C.

It has been found that the addition of a small amount of chelating agent to the intercapsular colored buffer solution increased the stability to at least 2 years or more. Suitable chelating agents include, for example, ethylene-diaminetetraacetic acid (“EDTA”), n-(2-hydroxyethyl)ethylenediamine-N,N′N″-triacetic acid (“HEDT”), triethanolamine, citric acid, nitrolotriacetic acid, and ethyleneglycol-bis (2-aminoethyl-N,N′,N″,N′″-tetraacetic acid (“EGTA”).

The effective amounts of chelating agent in the intercapsular colored buffer solution can vary from about 0.005% (w/v) to about 0.1% (w/v), preferably about 0.01% (w/v) to about 0.05% (w/v) and most preferably about 0.02%(w/v).

### EXAMPLE 1

Tap water usually contains contaminating metals such as calcium and iron. These metals accelerate the formation of peroxides in non-ionic surfactants such as Plurafac RA-20 (BASF Corporation). Peroxides are strong oxidizers and bleaching agents that form very reactive free radicals that can adversely affect the structure of phthalocyanine dyes and eliminate the dye color.

Four compositions were prepared as follows to evaluate the stability of the intercapsular colored buffer solution:

(a) Solution A—An intercapsular buffer solution was formulated with 0.02% Plurafac RA-20, 50 mM (0.2% w/v) sodium hydroxide and 0.44 ml (6.6 mg) Pontamine Brilliant Blue dye, and diluted with tap water to reach a total volume of one liter.

(b) Solution B—0.55 mM (0.02% w/v) EDTA tetrasodium salt hydrate was added to another intercapsular buffer formulation as in Solution A above and diluted with tap water to reach a total volume of one liter.

(c) Solution C—An intercapsular buffer solution was formulated with 50 mM sodium hydroxide, 0.02% Plurafac RA-20 and 6.6 mg/liter Pontamine Brilliant Blue dye and diluted with deionized water to reach a total volume of one liter.

(d) Solution D—0.55 mM (0.02% w/v) EDTA tetrasodium salt hydrate was added to another intercapsular buffer formulation as in Solution C above, and diluted with deionized water to reach a total volume of one liter.

The initial optical density values of the solutions were measured on a Cary 300 spectrophotometer (Varian Inc.) and their stability after 7, 16 and 36 days at 37° C. are shown in Table 1.

TABLE 1
Optical Density (“O.D.”)

| | 616 nm aggregate peak; 661 nm monomer peak or shoulder | | | |
|---|---|---|---|---|
| Day | Solution A | Solution B | Solution C | Solution D |
| 1 | 0.9707; 0.7531 | 1.0475; 0.7733 | 1.0604; 0.7830 | 1.0534; 0.7773 |
| 7 | 0.5741; shoul- | 1.0491; 0.7745 | 1.0571; 0.7785 | 1.0525; 0.7754 |
| | der (−41%) | (˜0%) | (˜0%) | (˜0%) |
| 16 | 0.5026; shoul- | 1.0479; 0.7719 | 1.0549; 0.7736 | 1.0505; 0.7718 |
| | der (−48%) | (˜0%) | (0.5%) | (−0.3%) |
| 36 | 0.3672; shoul- | 1.0518; 0.7714 | 1.0340; 0.7364 | 1.0515; 0.7699 |
| | der (−62%) | (˜0%) | (−2.5%) | (−0.1%) |

As can be seen in Table 1, Solution A which contained tap water and metal ions, caused a decrease in absorbance at 616 nm. The stability of the intercapsular buffer solution improved with Solution B with the addition of EDTA to the formulation even though tap water was used to dilute the formulation.

Solution C was formulated and diluted with deionized water containing no metal contaminants. The improved stability of Solution C was attributed to the replacement of tap water with deionized water which contains no metal contaminants.

The results demonstrate that elimination of metal ions in the water is necessary to establish good shelf stability for the dye in the intercapsular colored buffer solution.

The results also demonstrated that further increased stability of the colored buffer formulation was obtained with Solution D when EDTA, a metal chelator, was added to the formulation, and deionized water was used to dilute the colored buffer solution.

It was found that the aggregate peak at lower wavelength was nearly unaffected by the changes in the surrounding media introduced by the carryover effect of R1 and R2 or both R1 and R2 into the intercapsular buffer solution. Consequently this peak was used to assess the dilution caused by the carryover effect.

As noted earlier, the dilution of the dye in the intercapsular buffer solution caused by the carryover of reagents R1 and/or R2 was monitored on the ADVIA IMS clinical analyzer (Bayer Corporation) by measuring the absorbance. The aggregate wavelength of 616 nm was used because it is insensitive to changes in pH. Therefore, the dilution effect that results from carry-over of the R1/R2/sample into the intercapsular colored buffer solution can be measured. By using the peak at 616 nm, the absorbance measurement is not by changes in pH that occur when there is R1/R2/sample carry-over intercapsular colored buffer solution. It is only affected by the dilution ye caused by reagents R1 and/or R2.

What is claimed is:

1. A method for reducing erroneous results in a capsule chemistry liquid analysis system wherein a colorless intercapsular buffer solution B is used to separate test packages comprising sample S, reagent R1, and reagent R2 in an analytical line, and wherein small amounts of R1 and/or R2 become absorbed in the intercapsular buffer solution, comprising incorporating a dye in the colorless intercapsular buffer solution to form a colored intercapsular buffer solution, and measuring the amount of R1 and/or R2 absorbed into the colored intercapsular buffer solution by monitoring the change of the absorbance of the colored intercapsular buffer solution.

2. The method of claim 1, wherein the colorless intercapsular buffer solution comprises an aqueous solution of an alkali hydroxide and a surfactant.

3. The method of claim 2, where a phthalocyanine dye is added to the colorless intercapsular buffer solution to form the colored intercapsular buffer solution.

4. The method of claim 1, wherein the amount of dye varies from about 1 mg/liter to about 10 mg/liter.

5. The method of claim 3, wherein the phthalocyanine dye is a sulfonic acid phthalocyanine dye.

6. The method of claim 5, wherein the phthalocyanine dye is a copper phthalocyanine sulfonic acid dye.

7. The method of claim 1, wherein a chelating agent is added to the colored intercapsular buffer solution to reduce color degradation.

8. The method of claim 3, wherein the chelating agent is selected from the group consisting of ethylenediaminetetraacetic acid, n-(2-hydroxyethyl)ethylenediamine-N,N′N″-triacetic acid, triethanolamine, citric acid, nitrolotriacetic acid, and ethyleneglycol-bis (2-aminoethyl-N,N′,N″,N′″-tetraacetic acid.

9. The method of claim 2, wherein the amount of alkali hydroxide in the intercapsular buffer solution varies from about 0.01 M to about 0.1 M.

10. The method of claim 2, wherein the surfactant is selected from the group consisting of linear alcohol alkoxylates, and non-ionic alkylaryl polyether alcohols.

11. The method of claim 10, wherein the amount of surfactant varies from about 0.5% (w/v) to about 2.0% (w/v).

12. A colored intercapsular buffer solution composition adapted to reduce erroneous results from the absorption of small amounts of reagent R1 and/or R2 into the intercapsular buffer solution of a capsule chemistry liquid analysis system, comprising:
a) a phthalocyanine dye;
b) an alkali hydroxide;
c) a surfactant; and
d) a chelating agent.

13. The composition of claim 12, wherein the amount of phthalocyanine dye varies from about 1.0 mg/liter to about 10.0 mg/liter.

14. The composition of claim 12, wherein the alkali hydroxide is selected from the group consisting of sodium hydroxide, and potassium hydroxide.

15. The composition of claim 12, wherein the amount of alkali hydroxide varies from about 0.01 M to about 0.1 M.

16. The composition of claim 12, wherein the surfactant is selected from the group consisting of linear alcohol alkoxylates, and non-ionic alkylaryl polyether alcohols.

17. The composition of claim 12, wherein the surfactant varies from about 0.5 weight % to about 2.0 weight %.

18. The composition of claim 12, wherein the chelating agent is selected from the group consisting of ethylenediaminetetraacetic acid, n-(2-hydroxyethyl)ethylenediamine-N,N′N″-triacetic acid, triethanolamine, citric acid, nitrolotriacetic acid, and ethyleneglycol-bis (2-aminoethyl-N,N′,N″,N′″-tetraacetic acid.

19. The composition of claim 12, wherein the amount of chelating agent varies from about 0.01 weight % to about 0.1 weight %.

20. The composition of claim 12, wherein the phthalocyanine dye is a sulfonic acid phthalocyanine dye
